(19) United States
(12) Patent Application Publication
(10) Pub. No.: US 2023/0151317 A1
(43) Pub. Date: May 18, 2023

(54) In Vitro Manufacturing And Purification Of Therapeutic mRNA

(71) Applicant: Nature's Toolbox, Inc., Rio Rancho, NM (US)

(72) Inventors: Michael Humbert, Santa Fe, NM (US); Alexander Koglin, Santa Fe, NM (US); Charlie Villanueva, Albuquerque, NM (US); Matthias Strieker, Santa Fe, NM (US)

(21) Appl. No.: 17/913,392

(22) PCT Filed: Apr. 16, 2021
(86) PCT No.: PCT/US21/27774

§ 371 Date: Sep. 21, 2022

Related U.S. Application Data
(60) Provisional application No. 63/011,133, filed on Apr. 16, 2020.

(51) Int. Cl. C12M 1/40; C12P 19/34; C12N 9/12; C12M 1/26; C12M 1/00

(52) U.S. Cl. CPC C12M 21/18; C12P 19/34; C12N 9/1241; C12M 33/14; C12M 47/12

(57) ABSTRACT

The invention includes novel, systems, methods and compositions for the in vitro production of polynucleotides, and in particular the production of mRNA for use in therapeutic applications.

This International PCT Application claims the benefit of and priority to U.S. Provisional Application No. 63/011,133, filed Apr. 16, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 16, 2021, is named “90125-00181-Sequence-Listing-AF.txt” and is 26.9 Kbytes in size.

### TECHNICAL FIELD

The invention generally relates to the field of in vitro production of polynucleotides, and in particular the production of mRNA for use in therapeutic applications.

### BACKGROUND

Messenger RNA (mRNA) is the template molecule that is transcribed from cellular DNA and is translated into an amino acid sequence, i.e., a protein, at ribosomes in the cells of an organism. In order to control the expression level of the encoded proteins, mRNAs possess untranslated regions (UTRs) flanking the actual open reading frame (ORF) which contains the genetic information encoding the amino acid sequence. Such UTRs, termed the 5′-UTR and the 3′-UTR, respectively, are sections of the mRNA located before the start codon and after the stop codon. Further, mRNA contains a poly(A) tail region which is a long sequence of adenine nucleotides which promotes export of mRNA from the nucleus, translation and to some extent protects the mRNA from degradation. Scientific and technological advances of the recent years have made mRNA a promising candidate for a variety of uses, including diagnostic applications, and therapeutic products, like vaccines.

Due to the increasing demands of the medical community to enable personalized medicine, but also emergency responses in epidemic crisis situations, such as with the recent COVID-19 pandemic, many approaches have been developed for mRNA production at scale. Most current methods utilize fermentation to synthesize mRNA in culture from self-replicating DNA templates, then isolate the total RNA as raw material utilizing volatile organic solvents. These processes are costly, dangerous, produce hazardous waste streams that must be mediated, while the production rate is severely dependent on the performance of the producing strain and the ability to remove impurities from diverse tRNA, rRNA and host mRNA.

As a can be seen, there exist a long-felt need for an effective in vitro mRNA manufacturing process that does not require volatile organic solvents, produces no hazardous waste stream, and costs significantly less than its fermentation-based counterpart, while generating uniform pure mRNA fit for therapeutic applications.

### SUMMARY OF THE INVENTION

One aspect of the current invention includes novel in vitro methods for the production of polynucleotides, and in particular mRNA that may be directed to one or more diagnostic or therapeutic applications. In one preferred aspect, the invention includes a fully recombinant stable, reliable and functional in vitro system for the batch, or continuous flow production of polynucleotides, and preferably RNA. In this preferred aspect, the current improved in vitro system may generate an in vitro environment configured to mimic the production of polynucleotides that occurs in vivo by utilizing the components that are involved in polynucleotide transcription.

Another aspect of the current invention includes novel in vitro methods for the production of mRNA. In this embodiment, an in vitro bioreactor, and preferably a batch or continuous-flow bioreactor may be configured to combine isolated RNA Polymerase (RNAP) and a nucleotide template, and preferably a linear, non-self-replicating DNA template, along with a plurality of Nucleotide Triphosphates (NTPs) which may be incorporated into the synthesized mRNA molecules through the action of the RNAP, and an energy source, such as the novel inorganic polyphosphate energy-regeneration system generally described by Koglin and Humbert in PCT Application No. PCT/US2018/012121, the description, figures, examples, sequences and claims being incorporated herein by reference in their entirety). In a preferred embodiment, the synthesized mRNA can then be purified and used in a variety of downstream purposes, including diagnostic or therapeutic uses, such as vaccines directed to select target pathogens.

In a preferred embodiment, the production of macromolecules using the recombinant cell-free system of the invention may be accomplished in a bioreactor system. As used herein, a “bioreactor” may be any form of enclosed apparatus configured to maintain an environment conducive to the production of macromolecules, and preferably polynucleotide macromolecules, and even more preferably mRNA transcribed from a DNA template in vitro. A bioreactor may be configured to run on a batch, continuous, or semi-continuous basis, for example by a feed, or feeder solution. In one aspect, the invention may further include a bioreactor configured to produce mRNA. In this aspect, the present invention may be particularly suited for operation with a continuous-flow bioreactor system that may include one or more hollow continuous-flow conduits, for example made of a fibrous material in fluid communication with an external bioreactor compartment. In this aspect, the hollow continuous-flow conduits form as an exchange medium for in vitro transcription or polynucleotides, and preferably mRNAs.

Another aspect of the invention includes the synthetic biological production of polynucleotides, and preferably mRNA through a variety of reaction types. In one embodiment, a batch reaction may be used to produce mRNAs in vitro. In this preferred embodiment, an isolated RNAP, DNA template, and NTPs may all combine into a batch-fed reaction chamber and incubated until the reaction is exhausted. To circumvent the scaling limitation of the batch reaction, in another preferred embodiment, a continuous-flow bioreactor may be used to produce mRNAs in vitro. A continuous-flow bioreactor may be configured to operate continuously, such that it may be infused with new input materials while producing large amount of mRNA that can be output during or after the reaction process.

Another aspect of the invention may include novel systems and methods for the isolation and purification of polynucleotides, and preferably mRNA produced in the in vitro production system of the invention. In a preferred embodiment, the reaction output containing the synthesized mRNA may undergo a step-wise isolation and purification process that allows for the sequential removal of reaction mixture components, namely RNAP, template DNA, free NTPs and buffer from the mRNA output without the use of hazardous organic solvents or costly disposable purification kits. In one embodiment, the invention may utilize a purification column cascade, in which the reaction material from either batch or continuous-flow bioreactor are washed over a protein-affinity resin, followed by a DNA affinity resin or vice versa. This step may remove all protein and DNA template reaction components except for the free nucleotides that were unreacted. The free NTPs and buffer may be removed by alcohol precipitation, leaving only the isolated, precipitated mRNA material. The mRNA can then be resolubilized for the desired downstream application or dried for long-term storage or reduced volume delivery.

Another aspect of the invention may include pharmaceutical compositions for a novel mRNA-based vaccine directed to a target pathogen, such as COVID-19, produced by one or more of the in vitro mRNA production methods described herein, as well as their therapeutic use for the treatment of subjects in need thereof.

Another aspect of the invention may include novel systems, methods, and compositions for the multi-staged in vitro production of mRNA. In one preferred aspect, mRNA production from a DNA template is de-coupled from the addition of the poly(A) tail, or poly(A) tailing. For example, mRNA production from a DNA template in a first bioreactor, and preferably a continuous-flow bioreactor of the invention. This first stage production step production may or may not be coupled with the addition of a 5′ cap to the mRNA transcript. In a second stage, the mRNA transcript may be introduced to a second bioreactor, and preferably a continuous-flow bioreactor of the invention and may be further modified to include a poly(A) tail. Optionally, the mRNA may be modified to include a 5′ prime cap—assuming it was not included in the first bioreactor as noted above.

Another aspect of the invention may include novel systems, methods, and compositions for the multi-staged in vitro production of mRNA. In one preferred aspect, mRNA production from a DNA template is de-coupled from the addition of the poly(A) tail, or poly(A) tailing. For example, mRNA production from a DNA template in a first bioreactor, and preferably a continuous-flow bioreactor of the invention. This first stage production step production may or may not be coupled with the addition of a 5′ cap to the mRNA transcript. In a second stage, the mRNA transcript may be introduced to a second bioreactor, and preferably a continuous-flow bioreactor of the invention and may be further modified to include a poly(A) tail. Optionally, the mRNA may be modified to include a 5′ prime cap—assuming it was not included in the first bioreactor as noted above. This multi-stage in vitro production of mRNA has several advantage. For example, an operator can limit the unwanted synthesis of double stranded RNA (dsRNA), since the post-transcriptional generation of the poly(A) tail is decoupled from the RNA synthesis. The multi-stage production system of the invention also reduces the manual handling of the mRNA, further and reduce the risk of sheering. Finally, the multi-stage production system of the invention enables a one-step isolation/purification of the mRNA, for example via a polydT resin binding poly(A)-tailed RNA only.

Another aspect of the invention may include novel systems, methods, and compositions for the multi-staged in vitro production of mRNA having a Cap1 enzymatic processing system. In one preferred aspect, a Cap0/Cap1 enzymatic processing system may operate as a checkpoint to recognize the Cap0 on the RNA before enzymatic methylation generates a Cap1 followed by production of a poly(A) tail. This selection for the poly(A) tail ensures that the RNA is also fully capped during production resulting in improved yields of fully-functional mRNAs.

Another aspect of the invention may include novel multi-staged in vitro production of mRNA wherein mRNA synthesis and poly(A) tailing are decoupled. In this aspect, a reaction mixture having a DNA template, such as a linearized plasmid, PCR product, amine-function surface-tethered PCR product, a first quantity of RNA polymerase and reaction buffer is introduced to a first bioreactor, which may include a batch or continuous-flow bioreactor of the invention, or other appropriate bioreactor, such as a hollow fiber reactor as described herein. In a preferred embodiment, the reaction mixture is introduced to, and passes through the inner reaction cell of the first bioreactor. This inner reaction cell may be in fluid communication with a feed chamber holding a feed solution containing nucleotides NTP, reaction buffer, which may include components necessary to catalyze and drive mRNA synthesis as noted herein, and pyrophosphatase, which catalyzes the hydrolysis of pyrophosphate to inorganic phosphate. In this embodiment, the feed solution may be circulated in a countercurrent flow as compared to the reaction mixture. In this embodiment, the feed chamber and reaction cell may be configured to include a continuous-flow aspect as described generally herein.

As noted above, the compartmentalization and counterflow of the feed solution and reaction mixture creates a gradient such that the free NTs from the feed solution, when they pass through the feed chamber, may be drawn into the internal compartment of the reaction cell where they may react with the components of the reaction mixture. In this embodiment, an RNAP may associate with a DNA template having a target sequence that encodes a target mRNA, and enzymatically catalyze the incorporation of NTs into a target mRNA nucleotide.

The reaction mix from the inner reaction cell, after undergoing one or more reaction cycles, may be extracted and introduced to a mixing cell, where the newly synthesized mRNA is diluted, for example to a ratio of 1:5 in with a buffer, and preferably a high salt buffer. Dilution and addition of the high salt buffer generates a highly concentrated RNA solution and inactivates the RNAP and further adjusts the buffer conditions to enable the enzymatic activity of a poly(A) polymerase during the second stage poly(A) tailing step.

In a preferred embodiment, enzymes for 5′ capping and poly(A) tailing of the mRNA transcript may also provide in the dilution buffer. This new reaction mix is then introduced into the reaction cell of a second hollow fiber reactor having a feed chamber containing an additional components, such as nucleotide triphosphates, such as ATP, and GTP as well S-adenosylmethionine (SAM). As noted above, these components may be positioned within the feed chamber forming a gradient with the reaction cell such that they pass through a porous barrier, such as a hollow fiber barrier (34) as described herein, and into the reaction cell and act as substrates for 5′ capping and poly(A) tailing of the mRNA transcript. In this configuration, the mRNA is capped and poly(A) tailed, the reaction mix from the reaction cell of the second hollow fiber reactors is diluted in a high salt buffer to enable the slow binding of the poly(A) tailed RNA to a polydT resin. The other components pass over the resin and are collected. In certain embodiment, recombinant enzymes from the reaction mixture are captured in a column by an affinity resin before being washed over the polydT resin. After the reaction mix is completely washed over the polydT resin and poly(A) modified RNA is bound to the resin, the resin is flow washed with a high salt buffer and afterwards the washed and poly(A)-modified RNA is released from the resin with distilled water. The final product of the multi-staged in vitro production system is concentrated capped and poly(A) tailed RNA in distilled water, which may further be subject to additional processing, such as encapsulation in lipid nanoparticles (LNPs) for therapeutic applications.

Additional aspects of the inventive technology will become apparent from the specification, figures and claims below.

### BRIEF DESCRIPTION OF THE FIGURES

Aspects, features, and advantages of the present disclosure will be better understood from the following detailed descriptions taken in conjunction with the accompanying figures, all of which are given by way of illustration only, and are not limiting the presently disclosed embodiments, in which:

FIG. 1: Shows a schematic of a system of continuous flow mRNA production system in one embodiment thereof.

FIG. 2: Shows a schematic of a system of mRNA batch production system in one embodiment thereof.

FIG. 3: Shows a schematic of a system of mRNA purification and isolation in one embodiment thereof.

FIG. 4: Shows an exemplary 1.0% agarose gel that demonstrate mRNA production reactions conducted on Mar. 30, 2020, 4/1/2020, and 4/3/2020. A marker ladder is loaded on lane 1, purchased from New England Biolabs (NEB). A total of 10 ul of the unpurified reaction (bulk) and purified reactions from 3/30/2020 are loaded on the gel on lanes 2 and 3. A total of 10 ul of the unpurified reaction (bulk) and purified reactions from 4/1/2020 are loaded on the gel on lanes 4 and 5. Lanes 6-11 show 10 ul samples taken from the purification process described generally herein. Lane 6 is the unpurified reaction (bulk); lane 7 is the material after the reaction mixture is applied to a protein affinity column; lane 8 shows the product after the reaction mixture is applied to a DNA affinity column; lane 9 shows the reaction mixture after it has undergone alcohol precipitation and washed, then subsequent solubilized in sterile water; lane 10 shows the precipitated and isolated product after it is sterile filtered; Lane 10 shows the precipitated and isolated MRNA product after it is purified through a RNA purification column (provided by NEB) and optionally concentrated. The gel is visualized by illumination on a UV table at 365 nm.

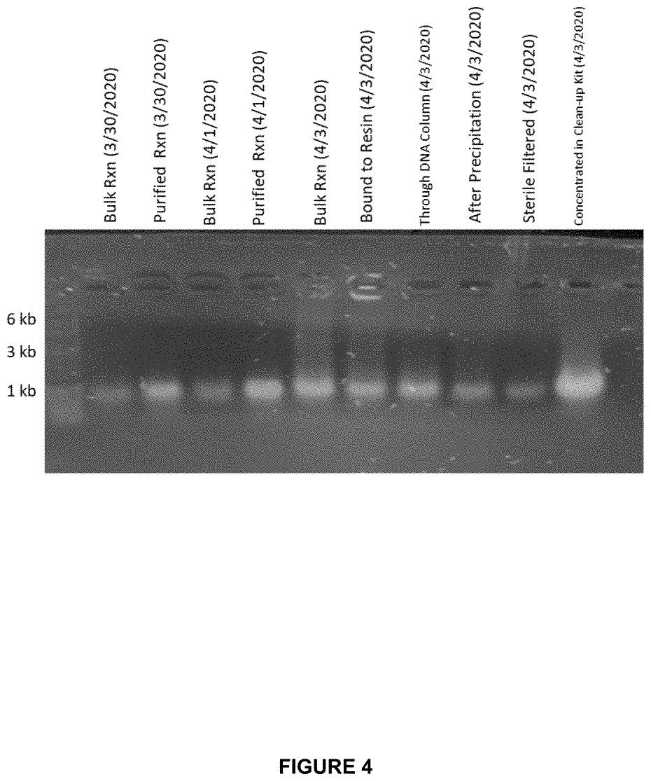

FIG. 5A: shows schematics of a system of multi-Stage mRNA production system in one embodiment thereof.

FIG. 5B: shows schematics of a system of multi-Stage mRNA production system utilizing a plurality of hollow fiber bioreactors in one embodiment thereof.

FIG. 6: Bioanalyzer Analysis. Three different mRNA production samples were capped and tailed using the system outlined in FIG. 5 and before/after samples were run an Agilent Bioanalyzer 2100 instrument using the Agilent RNA 6000 Nano kit according to the manufacturer's instructions. Analysis of the samples shows an apparent visual size shift through tailing and a minor population of untailed mRNA. 200 ng of raw vs capped and tailed mRNA on Bioanalyzer 2100. Expected row sizes: m001˜1650 nt, m002˜1675 nt, m003˜2600 nt, and tailing adds around 250-300 nt.

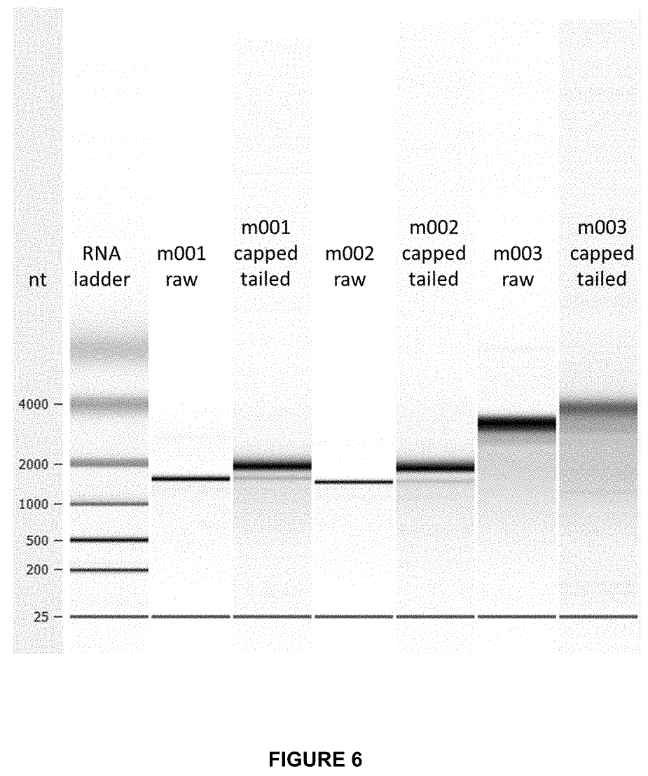

FIG. 7: Denaturing agarose gel analysis. In-vitro transcribed, large (11000 nt) mRNA was produced according the system described in FIG. 5, purified and analyzed on denaturing agarose gel. 1% agarose was melted and used with 2.5% formaldehyde and 0.01% gel red to pour a denaturing gel to analyze long mRNA samples. 1 μg mRNA sample was run side-by-side with a commercial RNA ladder (RiboRuler HR). The denaturing gel prevents formation of most secondary structures but still a small smear of various mRNA populations will be visible. The successful production of such a large mRNA species shows the feasibility of the inventive system described herein.

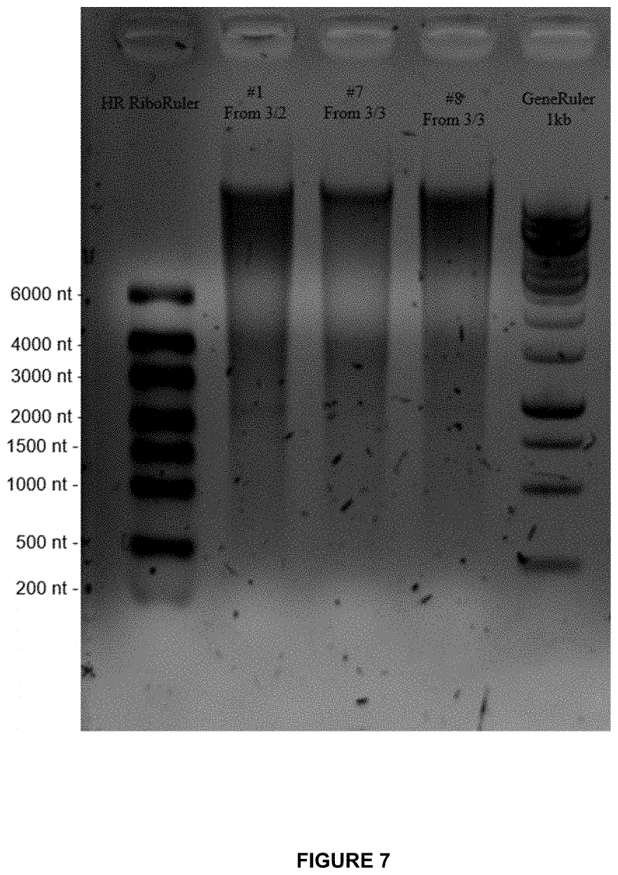

### DETAILED DESCRIPTION OF THE INVENTION

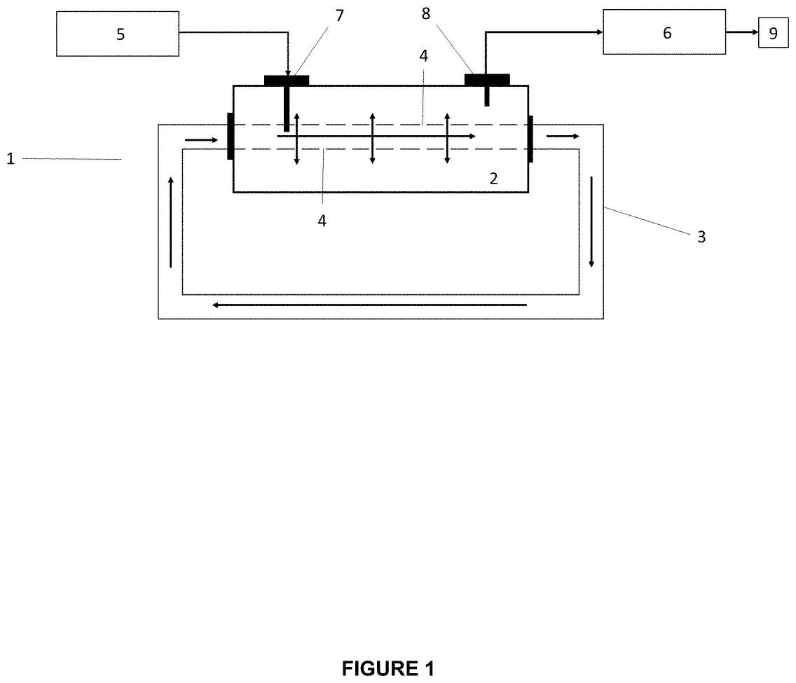

Generally referring to FIG. 1, in one embodiment the inventive technology may include a novel continuous-flow bioreactor (1) configured for the scaled in vitro production of polynucleotides, and in particular mRNA. Generally referring to FIG. 1 a continuous-flow conduit (3) may pass through the continuous-flow reaction chamber (2). In this embodiment, the continuous-flow conduit (3) may allow for the continuous recirculation of a feed solution which may include one or more of the following components:
a first quantity of isolated NTPs;
a quantity of a reaction buffer;
optionally the components of an inorganic polyphosphate energy-regeneration system as described in PCT/US2018/012121; and
optionally one or more co-factors for the production of mRNA polynucleotides.

In this embodiment, a continuous-flow reaction chamber (2) may hold a reaction mixture for the production of polynucleotides, and in particular mRNA. This input reaction mixture may include one or more of the following components:
a first quantity of isolated RNAP enzyme;
a first quantity of a DNA template;
a quantity of a reaction buffer;
optionally an initial quantity of isolated NTPs.

Notably, the continuous-flow conduit (3) may be in fluid communication with the continuous-flow reaction chamber (2). Specifically, as shown in FIG. 1, continuous-flow conduit (3) may be in fluid communication with the continuous-flow reaction chamber (2) through a plurality of conduit apertures (4) configured to allow one or more components of the feed solution to pass from the plurality of conduit apertures (4) and into the continuous-flow conduit (3). In one preferred embodiment, continuous-flow conduit (3) may include a continuous-flow conduit (3) made from hollow fibers. In this embodiment, continuous-flow conduit (3) can be made from MWCO PES membrane having pores between 5 kDa or 20 kDa in size. This fibrous membrane may further be treated to reduce protein and nucleotide binding. This treatment may include RNA-free and acetylated BSA deposited on the outside of the conduit, with the purified RNA polymerase positioned on the inside of the conduit where mRNAs may be produced. In this configuration, the plurality of conduit apertures (4) generates a gradient such that the free NTs from the feed solution when they pass through the continuous-flow reaction chamber (2) may be drawn into the internal compartment of the continuous-flow reaction chamber (2) where they may react with the components of the reaction mixture. In this embodiment, an RNAP may associate with a DNA template, and preferably a linear DNA template having a target sequence that encodes a target mRNA (14), and enzymatically catalyze the incorporation of NTPs into a target mRNA (14) nucleotide.

In certain embodiments, the target mRNA (14) may be configured to generate a three-dimensional structural shape, such as dsRNA configuration, of hairpin configuration that may be used to induce an RNA interference pathway in a subject in need thereof, while in additional embodiments the target mRNA (14) may be used as a pharmaceutical composition. For example, in one embodiment, the target mRNA (14) may encode an antigenic peptide, for example to a viral protein. In this embodiment, the mRNA may be administered to a subject in need thereof and be translated to form the antigenic protein that may, in turn elicit an immune response.

For example, in one preferred embodiment, the continuous-flow bioreactor (1) may be configured to produce mRNAs encoding one or more antigenic peptides directed to elicit an immune response directed to COVID-19 coronavirus in a subject in need thereof. More specifically, in this embodiment, the continuous-flow bioreactor (1) may be configured to produce mRNAs encoding one or more multi-valent COVID-19 coronavirus constructs described by Koglin and Humbert in U.S. Application No. 62/992,072, the specification, figures, sequences and construct configurations being specifically incorporated herein by reference.

In some embodiments, at least one coding region of the mRNA produce according to the invention encodes at least two, three, four, five, six, seven, eight or more antigenic peptides or proteins comprising or consisting of COVID-19 coronavirus protein, or a fragment or variant thereof. More preferably, at least one coding region encodes at least two, three, four, five, six, seven, eight or more antigenic peptides or proteins comprising or consisting of a spike protein subunit 1 (S1), ii) the receptor-binding motif (RBM) of S1; and ii) the nucleocapsid protein (NCP), as well as fragments, or variants of the same of a COVID-19 coronavirus, or a fragment or variant of any one of these proteins, which may further be coupled with a signal peptide, and preferably an IgE signal peptide (incorporated SEQ ID. NO 9). Even more preferably, at least one coding region encodes at least two, three, four, five, six, seven, eight or more amino acid sequences selected from the group consisting of incorporated SEQ ID NO: 1-6 In, or a fragment or variant of any one of these amino acid sequences. Notably, incorporate sequences are identified with an “In” designation.

Referring again to FIG. 1, the components of the reaction mixture may be loaded into the internal compartment of the continuous-flow reaction chamber (2) prior to the initiation of the reaction or may be replenished during operation as may be desired. The feed solution may also be loaded into the continuous-flow conduit (3) prior to initiation of the reaction. For example, as shown in FIG. 1, a feed solution may be added to the continuous-flow conduit (3) from an input reservoir (5) coupled with an input valve (7) configured to allow real-time injection of feed solution into the continuous-flow conduit (3). In certain embodiments, the addition of feed solution may be accomplished manually, while in alternative embodiments the processes may be automated. In this latter embodiment, feed solution may be added to the system based on predetermined schedule, or based on a pre-determined threshold, such as concentration of NTPs in the feed solution, concentration of mRNAs synthesized in the continuous-flow reaction chamber (2), or another parameter such as energy consumption of the reaction and the like. Such parameters may be measured by one or more sensors in communication with a computer system configured to run a computer-executable program in response to a change in one or more parameters as generally described herein.

Notably, after initiation of the reaction in the internal compartment of the continuous-flow reaction chamber (2), as NTPs are incorporated into the newly synthesized mRNA, fresh NTPs, among other factors may be continuously provided to the continuous-flow conduit (3). Such metered-production allows for a more efficient use of the reaction enzymes and energy usage, for example in the form of the energetic hydrolysis of nucleotide triphosphates, such a one or more nucleotide tri-phosphates selected from the group consisting of: adenine triphosphate (ATP); guanosine triphosphate (GTP), Uridine triphosphate (UTP), and Cytidine triphosphate (CTP).

The continuous-flow bioreactor (1) of the invention may further be configured to include an inorganic polyphosphate energy-regeneration system generally comprising a cellular adenosine triphosphate (ATP) energy regeneration system. In this embodiment, the continuous-flow reaction chamber (2) may include:
a quantity of isolated Adenosyl Kinase enzyme, and preferably Gst AdK from a thermophilic bacteria;
a quantity of isolated Polyphosphate Kinase enzyme Taq PPK from a thermophilic bacteria;
a quantity of inorganic polyphosphate (PPi) from a thermophilic bacteria; and
a quantity of adenosine monophosphate (AMP);

In this embodiment, the Adenosyl Kinase (AdK) and Polyphosphate Kinase (PPK) enzymes work synergistically to regenerate cellular ATP energy from PPi and AMP. More specifically, as generally shown in FIG. 8 of the '121 Application (incorporated herein by reference), in another preferred embodiment, isolated and purified Gst AdK (SEQ ID NO. 8 In of the '121 application incorporated herein by reference) and/or TaqPPK (SEQ ID NO. 11 In of the '121 application incorporated herein by reference) may be added to this cell-free expression system with a quantity of inorganic polyphosphate. In one embodiment, this quantity of inorganic polyphosphate may include an optimal polyphosphate concentration range. In this preferred embodiment, such optimal polyphosphate concentration range being generally, defined as the concentration of inorganic polyphosphate (PPi) that maintains the equilibrium of the reaction stable. In this preferred embedment, optimal polyphosphate concentration range may be approximately 0.2-2 mg/ml PPi.

As noted above, PPK can synthesize ADP from polyphosphate and AMP. In this preferred embodiment the coupled action of Gst AdK and PPK, may remove adenosine diphosphate (ADP) from the system by converting two ADP to one ATP and one adenosine monophosphate (AMP):

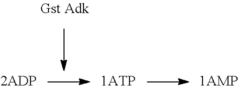

This reaction may be sufficiently fast enough to drive an equilibrium reaction of PPK towards production of ADP:

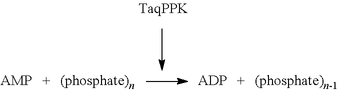

In this system, the presence of higher concentrations of AMP may further drive the TaqPPK reaction towards ADP.

An mRNA output (9) containing a portion of the reaction mixture and newly synthesized mRNAs in the continuous-flow reaction chamber (2) may be extracted for further modification. Referring again to FIG. 1, an mRNA output (9) may be extracted to the continuous-flow reaction chamber (2) to an output reservoir (6) coupled with an output valve (8) configured to allow extraction of the mRNA output (9) from the continuous-flow reaction chamber (2). In certain embodiments, the extraction of mRNA output (9) may be accomplished manually, while in alternative embodiments the processes may be automated. In this latter embodiment, mRNA output (9) may be extracted from the system based on predetermined schedule, or based on a pre-determined threshold, such as concentration of NTPs in the feed solution, concentration of mRNAs synthesized in the continuous-flow reaction chamber (2), or another parameter such as energy consumption of the reaction and the like. Such parameters may be measured by one or more sensors in communication with a computer system configured to run a computer-executable program in response to a change in one or more parameters as generally described herein.

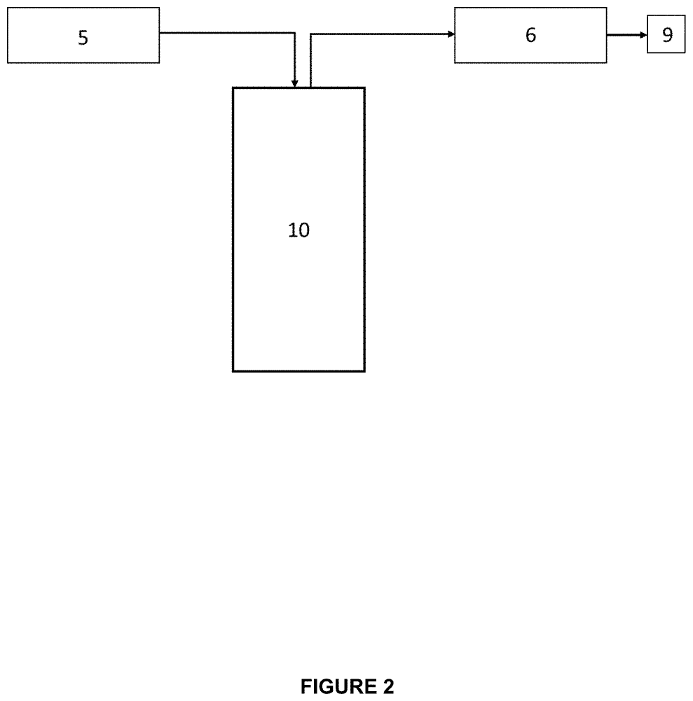

Generally referring now to FIG. 2, in one embodiment the inventive technology may include a novel batch-fed reaction chamber (10) configured for the scaled in vitro production of polynucleotides, and in particular mRNA. In this embodiment, a batch-fed reaction chamber (10) may hold a reaction mixture for the production of polynucleotides, and in particular mRNA. This input reaction mixture may include one or more of the following components:
a first quantity of isolated RNAP enzyme(s);
a first quantity of an isolated DNA template(s);
a quantity of a reaction buffer;
a first quantity of isolated NTPs;
optionally the components of an inorganic polyphosphate energy-regeneration system as described in PCT/US2018/012121; and
optionally one or more co-factors for the production of mRNA polynucleotides.

In this configuration, free RNAP may associate with a DNA template, and preferably a linear DNA template having a target sequence that encodes a target mRNA (14), and enzymatically catalyze the incorporation of NTPs into a target mRNA (14) nucleotide in the batch-fed reaction chamber (10). Again, referring to FIG. 1, one or more components of the reaction mixture may be placed in the batch-fed reaction chamber (10) through an input reservoir (5).

In certain embodiments, the target mRNA (14) generated in the batch-fed reaction chamber (10) may be configured to generate a three-dimensional structural shape, such as dsRNA configuration, of hairpin configuration that may be used to induce an RNA interference pathway in a subject in need thereof, while in additional embodiments the target mRNA (14) may be used as a pharmaceutical composition. For example, in one embodiment, the target mRNA (14) may encode an antigenic peptide, for example to a viral protein. In this embodiment, the mRNA may be administered to a subject in need thereof and be translated to form the antigenic protein that may, in turn elicit an immune response.

For example, in one preferred embodiment, the batch-fed reaction chamber (10) may be configured to produce mRNAs encoding one or more antigenic peptides directed to elicit an immune response directed to COVID-19 coronavirus in a subject in need thereof. More specifically, in this embodiment, the continuous-flow bioreactor (1) may be configured to produce mRNAs encoding one or more multi-valent COVID-19 coronavirus constructs described by Koglin and Humbert in U.S. Application No. 62/992,072, the specification, figures, sequences and construct configurations being specifically incorporated herein by reference.

In some embodiments, at least one coding region of the mRNA produce according to the invention encodes at least two, three, four, five, six, seven, eight or more antigenic peptides or proteins comprising or consisting of COVID-19 coronavirus protein, or a fragment or variant thereof. More preferably, at least one coding region encodes at least two, three, four, five, six, seven, eight or more antigenic peptides or proteins comprising or consisting of a spike protein subunit 1 (S1), ii) the receptor-binding motif (RBM) of S1; and ii) the nucleocapsid protein (NCP), as well as fragments, or variants of the same of a COVID-19 coronavirus, or a fragment or variant of any one of these proteins, which may further be coupled with a signal peptide, and preferably an IgE signal peptide (incorporated SEQ ID. NO 9). Even more preferably, at least one coding region encodes at least two, three, four, five, six, seven, eight or more amino acid sequences selected from the group consisting of incorporated SEQ ID NO: 1-6 In, or a fragment or variant of any one of these amino acid sequences.

Notably, after initiation of the reaction in the batch-fed reaction chamber (10), as NTPs are incorporated into the newly synthesized mRNA, the synthesizing reaction may proceed for a predetermined time, or until a threshold is met, such as a predetermined concentration of newly synthesized mRNAs, or until the enzymes, or energy source of the reaction mixture is expended. As noted above, in one embodiment, batch-fed reaction chamber (10) may include an energy source in the form of the energetic hydrolysis of nucleotide triphosphates, such as one or more nucleotide triphosphates selected from the group consisting of: Adenine triphosphate (ATP); Guanosine triphosphate (GTP), Uridine triphosphate (UTP), and Cytidine triphosphate (CTP). Finally, the batch-fed reaction chamber (10) of the invention may further be configured to include an inorganic polyphosphate energy-regeneration system as generally described above.

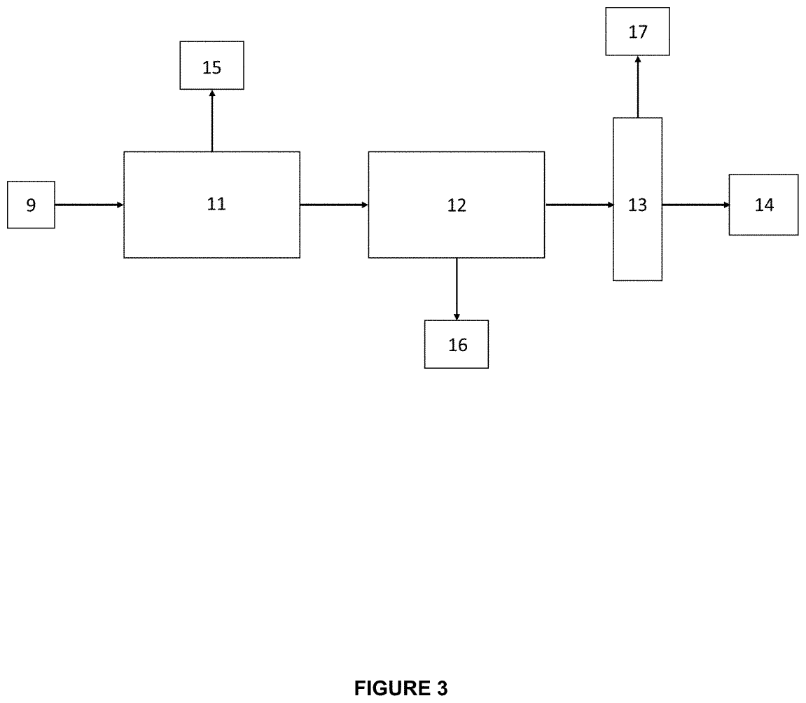

The inventive technology further includes systems and methods for the isolation of mRNA output (9) produced through either a batch-fed reaction chamber (10) system, or a continuous-flow bioreactor (1) as generally described above. Generally referring to FIG. 3, in one embodiment mRNA output (9) may pass through a protein affinity column (11) configured to capture the protein fraction (15) of the mRNA output (9) which may include the free RNAP or other proteins that may be present in the mRNA output (9). In the embodiment, the captured RNAP may be eluted from the protein affinity column (11) and reused in subsequence in vitro mRNA production reactions.

As further shown in FIG. 3, after removing the protein fraction (15), the mRNA output (9) may pass through a DNA affinity column (12) configured to capture the DNA fraction (16) of the mRNA output (9) which may include the free DNA templates that may be present in the mRNA output (9).

Again, as shown in FIG. 3, after removing the protein fraction (15) and DNA fraction (16), the mRNA output (9) may pass through a nucleotide precipitator (13) configured to remove the NTP fraction (17), as well as excess buffer or other components of the mRNA output (9). In a preferred embodiment, the free NTP fraction (17) may be separated and removed from the target mRNA (14) through one or more rounds of alcohol precipitation and washing to extract the target mRNA (14) that may be present in the mRNA output (9). The now isolated target mRNA (14) can be further purified, and optionally resolubilized for the desired downstream application or dried for long-term storage or reduced volume delivery. As noted above, the order of the protein affinity column (11) and DNA affinity column (12) may be switched, such that the DNA fraction (16) is removed first, while the protein fraction (15) is removed second and vice versa. Such order of operation is also applicable to the removal of the free NTP fraction (17).

The invention may include systems, methods and compositions for a hollow fiber bioreactor (20) which may be configured to the multi-stage production of mRNA as describe generally below. As shown in Figure, in a preferred embodiment, a hollow fiber bioreactor (20) may include a reaction cell (31) configured to contain a reaction mixture, preferably containing a DNA template, such as a linearized plasmid, PCR product, amine-function surface-tethered PCR product, a first quantity of RNA polymerase and reaction buffer and other components necessary for the in vitro production of mRNA. The mixing reaction cell (31) may be positioned within a feed chamber (32). This inner mixing reaction cell (31) may be in fluid communication with a feed chamber (32) holding, in embodiment, a feed solution containing nucleotides NTP, reaction buffer, which may include components necessary to catalyze and drive mRNA synthesis as noted herein, and pyrophosphatase, which catalyzes the hydrolysis of pyrophosphate to inorganic phosphate. As shown below, in a multi-staged system, a feed chamber (32) may contain a feed solution containing enzymes for 5′ capping and poly(A) tailing a mRNA transcript. As noted above, the components of the feed solution positioned within the feed chamber (32) may form a gradient with the reaction cell (32) such that they pass through a porous barrier, and into the reaction cell (32) and act as substrates for mRNA synthesis and/or 5′ capping and poly(A) tailing of the mRNA transcript.

In a preferred embodiment, the reaction cell (32) is separated from the feed chamber (32) by a porous barrier, which may preferably be a hollow fiber barrier (34) which may include MWCO PES membrane having pores between 5 kDa or 20 kDa in size. This fibrous membrane may further be treated to reduce protein and nucleotide binding. This treatment may include RNA-free and acetylated BSA deposited on the outside of the conduit, with the purified RNA polymerase positioned on the inside of the conduit where mRNAs may be produced.

As generally described above, the multi-stage in vitro production system (30) of the invention may include a plurality of bioreactors configured to decouple mRNA synthesis and poly(A) tailing, as well as optionally 5′ capping of the mRNA transcript. In this embodiment, mRNA is generated in a first bioreactor, while poly(A) tailing of the mRNA transcripts is performed in a second bioreactor. As further outline below, 5′ prime capping and modification of said cap may be performed concurrently with mRNA synthesis in the first bioreactor or may also be decoupled from the mRNA synthesis step and performed during poly(A) tailing in a second bioreactors.

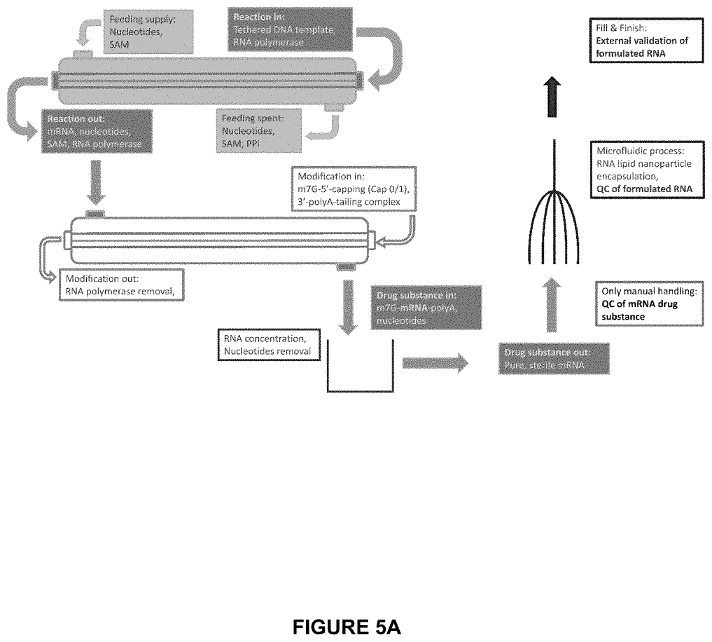

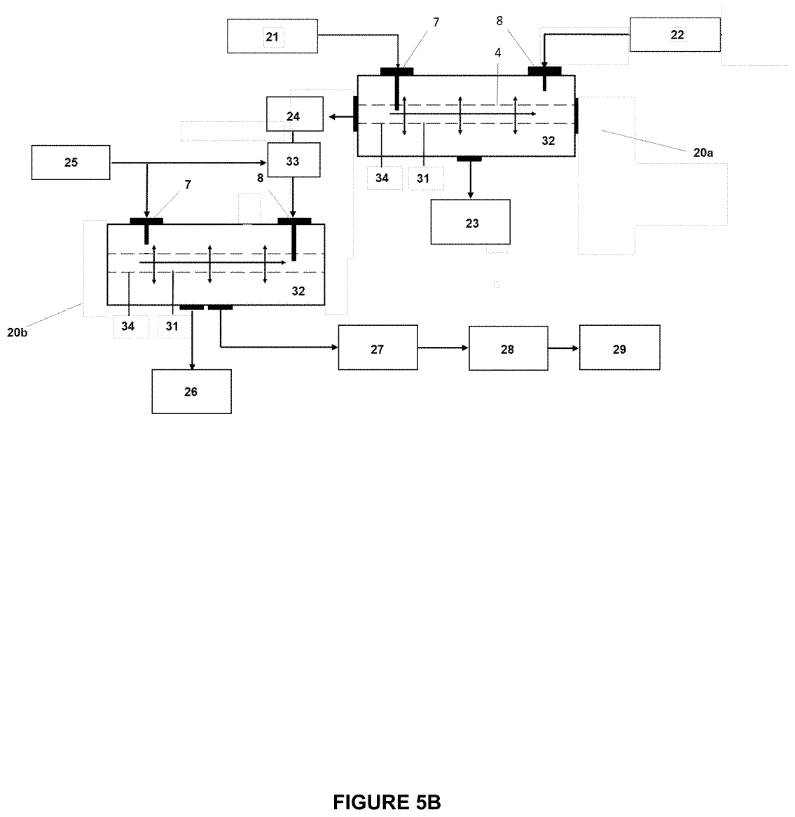

In the schematic flow-diagram provided in FIGS. 5A-B, the multi-stage in vitro production system (30) of the invention may include a first bioreactor, which in this embodiment is shown as a first hollow fiber reactor (20a) configured for the synthesis of mRNA macromolecules, and optionally 5′ capping of said transcript. Notably, the use of a first hollow fiber reactor (20a) is preferred, but not required as any in vitro mRNA bioreactor system may be used with the invention. Referring again to FIGS. 5A-B, first hollow fiber reactor (20a) may include a reaction cell (31) configured to contain a first stage reaction mixture (21), preferably containing a DNA template, such as a linearized plasmid, PCR product, amine-function surface-tethered PCR product, a first quantity of RNA polymerase (SEQ ID NO. 1), and reaction buffer. This reaction cell (31) of the first hollow fiber reactor (20a) may be in fluid communication with a feed chamber (32) holding a first stage feed solution (22) containing nucleotides NTP, reaction buffer, which may include components necessary to catalyze and drive mRNA synthesis as described or incorporated herein, and pyrophosphatase, which catalyzes the hydrolysis of pyrophosphate to inorganic phosphate. In this embodiment, the first stage feed solution (22) may be circulated in a countercurrent flow as compared to the first stage reaction mixture (21). In this embodiment, the feed chamber (32) and reaction cell (33) may individually, or collectively be configured for the continuous or batch inflow and outflow of components as described generally herein.

As noted above, the compartmentalization and counterflow of the first stage feed solution (22) and first stage reaction mixture (21) creates a gradient such that the free NTs from the first stage feed solution (22), when they pass through the feed chamber (32), may be drawn into the reaction cell (31) where they may react with the components of the first stage reaction mixture (21) such that an RNAP may associate with a DNA template having a target sequence that encodes a target mRNA (14), and enzymatically catalyze the incorporation of NTs into a target mRNA nucleotide forming a. This reaction cycle may be allowed to run for a pre-determined period of time, preferably 3-4 hour, or until a quantity of mRNA is produced in the reaction cell (31). Spent first stage feeding solution (23) may be extracted from the feed chamber (32) during or after the cycle is complete.

The second stage reaction mixture (24) containing mRNA transcripts lacking a poly(A) tail, and a 5′ cap in this embodiment may be extracted from the reaction cell (32) of the first hollow fiber bioreactor (20a) and introduced to a mixing cell (33) where the newly synthesized mRNA is diluted, preferably to a ratio of 1:5 in with a high salt buffer. Dilution and addition of the high salt buffer generates a highly concentrated RNA solution and inactivates the RNAP and further adjusts the buffer conditions to enable the enzymatic activity of a poly(A) polymerase during the second stage poly(A) tailing step.

Notably, in order to generate a mature mRNA ready for efficient translation by the ribosome it must contains two major modifications: a 5′ cap structure and a poly(A) tail. The m7G cap structure consists of a 7-methylguanosine triphosphate linked to the 5′ end of the mRNA via a 5′→5′ triphosphate linkage (m7G cap). The m7G cap, also known as cap 0 structure, is essential for the majority of protein translation in vivo. The m7G cap also protects the mature mRNA from degradation, allows for a regulated degradation mechanism, enhances pre-RNA splicing and directs nuclear export. In vivo, the cap 0 structure can be further modified to cap 1 structure by adding a methyl group to the 2′O position of the initiating nucleotide of the mRNA. The 2′O methylation in the cap 1 structure helps the mRNA evade innate immune response in vivo, making it especially important for mRNA's produced for therapeutic applications. More specifically, 5′ capping is the first step in co-transcriptional pre-mRNA processing and in many eukaryotes the capping machinery is directly bound to the phosphorylated C-terminal domain of RNAP. Biosynthesis of the cap 0 structure requires three consecutive enzymatic activities: hydrolysis of the 5′ triphosphate end of the nascent transcript to a diphosphate by an RNA triphosphatase; capping of the diphosphate with GMP by an RNA guanylyl-transferase, which uses GTP as a substrate and GMP covalently linked to an active site lysine as an intermediate; and finally, methylation of the 5′ guanine base at the N7 position by an RNA methyl-transferase (MT).

Referring again to FIGS. 5A-B, the modified reaction mixture (25) may be introduced to the second stage reaction mixture (24) in the mixing cell (33), or alternatively a second hollow fiber reactor (20b). As noted above, the modified reaction mixture (25) may contain optionally enzymes for 5′ capping, as well as specific enzymes for poly(A) tailing of the MRNA transcript. Additional enzymes may be added that initiate polyadenylation and extend the pol)A) tail. The second stage reaction mixture (24) may include a quantity of one or more of the following:
a mRNA triphosphatase enzyme;
a RNA guanylyl-transferase enzyme;
a mRNA methyltransferase enzyme;
a Poly(A) Polymerase enzyme;
a Poly adenylation initiator enzyme; and
a Poly adenylation extender enzyme.

In a preferred embodiment, second stage reaction mixture (24) may include a quantity of one or more of the following:
a mRNA triphosphatase enzyme according to the amino acid sequence of SEQ ID NO. 2;
a RNA guanylyl-transferase enzyme according to the amino acid sequence of SEQ ID NO. 3;
a mRNA cap guanine-N7 methyltransferase enzyme according to the amino acid sequence of SEQ ID NO. 4;
a Poly(A) Polymerase enzyme according to the amino acid sequence of SEQ ID NO. 5;
a Poly adenylation initiator enzyme according to the amino acid sequence of SEQ ID NO. 6; and
a Poly adenylation extender enzyme according to the amino acid sequence of SEQ ID NO. 7.

In this configuration, the cap1 generating methyltransferase (SEQ ID NO. 4), recognizes the cap0-GTP on the mRNA transcript, binds it and generates the cap1 methylation. The Poly(A) Polymerase recognizes and bind the cap1-methyltransferase, forming a complex and starts to generate the poly(A) tail such that the enzymes work cooperatively as a selection tool to ensure that all poly(A)-tailed RNA is also capped. Notably, the 5 enzyme required for the 5′ capping of the mRNA transcript may alternative be included in the first stage reaction mixture (21), with corresponding capping components, as described below being added to the first stage feed solution (22) such that the mRNA synthesis and 5′ capping is coupled within the first hollow fiber reactor (20a).

Again, referring to FIG. 5A-B, second stage reaction mixture (24) described above, of the mRNA transcript may also provide in the dilution buffer in the mixing cell (33), or alternatively into a second hollow fiber reactor (20b). This new solution is then introduced into a second bioreactor, and preferably the reaction cell (31) of a second hollow fiber reactor (20b) having a feed chamber (32) containing additional components, such as nucleotide triphosphates, such as ATP, and GTP as well S-adenosylmethionine SAM necessary for poly(A) tailing and 5′ capping, respectively. As noted above, these components may be positioned within the feed chamber (32) forming a gradient with the reaction cell such that they pass through a porous barrier, such as a hollow fiber barrier (34) as described herein, and into the reaction cell (31) and act as substrates for 5′ capping and poly(A) tailing of the mRNA transcript.

During one or more capping and pol(A) tailing cycles, the mRNA is capped, and poly(A) tailed. The spent modified reaction mixture (26) may be extracted from the hollow fiber reactor (20b), while the mRNA concentrate (27) from the reaction cell (31) of the second hollow fiber reactor (20b) may be extracted and introduced to a nucleotide removal apparatus (28) to remove any free nucleotides forming a purified mRNA (29) output. In this embodiment, the mRNA concentrate (27) from the reaction cell (31) of the second hollow fiber reactor (20b) may be diluted in a high salt buffer to enable the slow binding of the poly(A) tailed RNA to a polydT resin. The other components pass over the resin and are collected. In certain embodiments, recombinant enzymes from the reaction mixture are captured in a column by an affinity resin before being washed over the polydT resin. After the reaction mix is completely washed over the polydT resin and poly(A) modified RNA is bound to the resin, the resin is flow washed with a high salt buffer and afterwards the washed and poly(A)-modified RNA is released from the resin with distilled water. The final product of the multi-staged in vitro production system is concentrated capped, and poly(A) tailed RNA in distilled water (29), which may further be subject to additional processing, such as encapsulation in lipid nanoparticles (LNPs) for therapeutic applications.

Accordingly, in other preferred embodiments the target mRNA (14) is a purified or isolated mRNA. The terms “purified mRNA” or “isolated mRNA” are used interchangeably, as used herein has to be understood as mRNA which has a higher purity after certain purification steps, (such as alcohol purification, as well as for example HPLC, TFF, and other polynucleotide precipitation steps) than the starting material (e.g. in vitro transcribed mRNA in a continuous-flow bioreactor (1), or batch-fed reaction chamber (10). Typical impurities that are essentially not present in purified mRNA comprise peptides or proteins (e.g. enzymes derived from DNA dependent RNA in vitro transcription, e.g. RNA polymerases, RNases, BSA, pyrophosphatase, restriction endonuclease, DNase), spermidine, abortive RNA sequences, RNA fragments, free nucleotides (modified nucleotides, conventional NTPs, cap analogue), plasmid DNA fragments, buffer components (HEPES, TRIS, MgCI2) etc. Other impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full length RNA transcripts is as close as possible to 100%. Accordingly, “purified mRNA” or “isolated mRNA” as used herein has a degree of purity of more than 70%, 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

The term “dried mRNA” as used herein has to be understood as mRNA that has been lyophilized, or spray-dried, or spray-freeze dried as defined above to obtain a temperature stable dried mRNA (powder). It has to be understood also that “dried mRNA” as defined herein and “purified mRNA” as defined herein or “GMP-grade mRNA” as defined herein may have superior stability characteristics and improved efficiency (e.g. better translatability of the mRNA in vivo).

In further embodiments, the present invention provides a composition comprising the in vitro transcribed mRNA in a continuous-flow bioreactor (1), or batch-fed reaction chamber (10), when the mRNA encodes an antigenic peptide, and in particular a multi-valent COVID-19 mRNA vaccine, and at least one pharmaceutically acceptable carrier. In particular, the composition according to the invention comprises at least one mRNA, preferably as described herein, encoding at least one antigenic peptide or protein comprising or consisting of a spike protein subunit 1 (S1), ii) the receptor-binding motif (RBM) of S1; and ii) the nucleocapsid protein (NCP), as well as fragments, or variants of the same of a COVID-19 coronavirus, or from a fragment or variant of any one of these proteins.

The composition according to the invention is preferably provided as a pharmaceutical composition or as a vaccine. A “vaccine” is typically understood to be a prophylactic or therapeutic material providing at least one epitope of an antigen, preferably an immunogen. In some embodiment, the mRNA may encode a peptide having or providing at least one epitope. The term, “providing at least on epitope” means, for example, that the vaccine comprises the epitope (or antigen comprising or providing said epitope) or that the vaccine comprises a molecule that, e.g., encodes the epitope or an antigen comprising or providing the epitope. The antigen preferably stimulates the adaptive immune system to provide an adaptive immune response. The (pharmaceutical) composition or vaccine provided herein may further comprise at least one pharmaceutically acceptable excipient, adjuvant or further component (e.g. additives, auxiliary substances, and the like). In preferred embodiments, the (pharmaceutical) composition or vaccine according to the invention comprises a plurality or more mRNAs the in vitro transcribed mRNA in a continuous-flow bioreactor (1), or batch-fed reaction chamber (10), which may further comprise a multi-valent COVID-19 mRNA vaccine as described herein. According to another embodiment, the (pharmaceutical) composition or vaccine according to the invention may comprise an adjuvant, which is preferably added in order to enhance the immunostimulatory properties of the composition. In this context, an adjuvant may be understood as any compound, which is suitable to support administration and delivery of the composition according to the invention. Furthermore, such an adjuvant may, without being bound thereto, initiate or increase an immune response of the innate immune system, i.e. a nonspecific immune response. In other words, when administered, the composition according to the invention typically initiates an adaptive immune response due to an antigen as defined herein or a fragment or variant thereof, which is encoded by the at least one coding sequence of the inventive mRNA contained in the composition of the present invention. Additionally, the composition according to the invention may generate an (supportive) innate immune response due to addition of an adjuvant as defined herein to the composition according to the invention.

As with the (pharmaceutical) composition according to the present invention, the entities of the mRNA vaccine produced by one or more of the methods generally described above may be provided in liquid and or in dry (e.g. lyophilized) form. They may contain further components, in particular further components allowing for its pharmaceutical use. The mRNA, mRNA vaccine or the (pharmaceutical) composition of the same may, e.g., additionally contain a pharmaceutically acceptable carrier and/or further auxiliary substances and additives and/or adjuvants. mRNA, mRNA vaccine or the (pharmaceutical) composition of the same typically comprises a safe and effective amount of the mRNA according to the invention as defined herein, encoding an antigenic peptide or protein as defined herein or a fragment or variant thereof or a combination of antigens, preferably as defined herein. As used herein, “therapeutically effective amount” means an amount of the mRNA that is sufficient to significantly induce a positive immune response, that preferable prevent infection of COVID-19 coronavirus. At the same time, however, a “therapeutically effective amount” is small enough to avoid serious side-effects, that is to say to permit a sensible relationship between advantage and risk. The determination of these limits typically lies within the scope of sensible medical judgment. In relation to the mRNA, mRNA vaccine or the (pharmaceutical) composition of the present invention, the expression “therapeutically effective amount” preferably means an amount of the mRNA, mRNA vaccine or the (pharmaceutical) composition of the same that is suitable for stimulating the adaptive immune system in such a manner that no excessive or damaging immune reactions are achieved but, preferably, also no such immune reactions below a measurable level. Such a “therapeutically effective amount” of the mRNA of the (pharmaceutical) composition or vaccine as defined herein may furthermore be selected in dependence of the type of mRNA, e.g. monocistronic, bi- or even multicistronic mRNA, since a bi- or even multicistronic mRNA may lead to a significantly higher expression of the encoded antigen(s) than the use of an equal amount of a monocistronic mRNA. A “therapeutically effective amount” of the an mRNA, mRNA vaccine or the (pharmaceutical) composition of the same as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The mRNA, mRNA vaccine or the (pharmaceutical) composition of the same according to the invention can be used according to the invention for human and also for veterinary medical purposes, as a pharmaceutical composition or as a vaccine.

In a preferred embodiment, the mRNA of the (pharmaceutical) composition, and preferably a multi-valent COVID-19 mRNA vaccine or kit of parts according to the invention is provided in lyophilized form. Preferably, the lyophilized mRNA is reconstituted in a suitable buffer, advantageously based on an aqueous carrier, prior to administration, e.g. Ringer-Lactate solution, which is preferred, Ringer solution, a phosphate buffer solution. In a preferred embodiment, the (pharmaceutical) composition, the vaccine or the kit of parts according to the invention contains at least one, two, three, four, five, six or more mRNAs, preferably mRNAs which are provided separately in lyophilized form (optionally together with at least one further additive) and which are preferably reconstituted separately in a suitable buffer (such as Ringer-Lactate solution) prior to their use so as to allow individual administration of each of the (monocistronic) mRNAs. The vaccine or (pharmaceutical) composition according to the invention may typically contain a pharmaceutically acceptable carrier. The expression “pharmaceutically acceptable carrier” as used herein preferably includes the liquid or non-liquid basis of the inventive vaccine. If the inventive vaccine is provided in liquid form, the carrier will be water, typically pyrogen-free water; isotonic saline or buffered (aqueous) solutions, e.g., phosphate, citrate etc. buffered solutions. Particularly for injection of the inventive vaccine, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCI, Nal, NaBr, a2C(¼, NaHCCh, a2S0₄, examples of the optional potassium salts include e.g. KCI, KI, KBr, K2CO3, KHCO3, K2SO4, and examples of calcium salts include e.g. CaCb, Cal2, CaBr₂, CaCC>3, CaSC, Ca(OH)₂. Furthermore, organic anions of the aforementioned cations may be contained in the buffer. According to a more preferred embodiment, the buffer suitable for injection purposes as defined above, may contain salts selected from sodium chloride (NaCI), calcium chloride (CaCb) and optionally potassium chloride (KCI), wherein further anions may be present additional to the chlorides. CaCb can also be replaced by another salt like KCI. Typically, the salts in the injection buffer are present in a concentration of at least 50 mM sodium chloride (NaCI), at least 3 mM potassium chloride (KCI) and at least 0.01 mM calcium chloride (CaCb). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. in “in vivo” methods occurring liquids such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds may be used as well, which are suitable for administration to a person. The term “compatible” as used herein means that the constituents of the inventive vaccine are capable of being mixed with the mRNA according to the invention as defined herein, in such a manner that no interaction occurs, which would substantially reduce the pharmaceutical effectiveness of the inventive vaccine under typical use conditions. Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, com oil and oil from theobroma; polyols, such as, for example, polypropylene glycol, glycerol, sorbitol, mannitol and polyethylene glycol; alginic acid.

The choice of a pharmaceutically acceptable carrier is determined, in principle, by the manner, in which the pharmaceutical composition or vaccine according to the invention is administered. The composition or vaccine can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections. More preferably, composition or vaccines according to the present invention may be administered by an intradermal, subcutaneous, or intramuscular route, preferably by injection, which may be needle-free and/or needle injection. Compositions/vaccines are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or vaccine is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

For the sake of clarity and readability, the following scientific background information and definitions are provided. Any technical features disclosed thereby can be part of each and every embodiment of the invention. Additional definitions and explanations can be provided in the context of this disclosure.

Poly (A) sequence; A poly-A-tail also called “3′-poly(A) tail or poly(A) sequence” is typically a long sequence of adenosine nucleotides of up to about 400 adenosine nucleotides, e.g., from about 25 to about 400, preferably from about 50 to about 400, more preferably from about 50 to about 300, even more preferably from about 50 to about 250, most preferably from about 60 to about 250 adenosine nucleotides, added to the 3 end of a RNA. Moreover, poly(A) sequences, or poly(A) tails may be generated in vitro by enzymatic polyadenylation of the RNA, e.g., using Poly(A)polymerases derived from E. coli or yeast.

Polyadenylation: Polyadenylation is typically understood to be the addition of a poly(A) sequence to a nucleic acid molecule, such as an RNA molecule, e.g., to a premature mRNA. Polyadenylation may be induced by a so called polyadenylation signal. This signal is preferably located within a stretch of nucleotides at the 3′-end of a nucleic acid molecule, such as an RNA molecule, to be polyadenylated. A polyadenylation signal typically comprises a hexamer consisting of adenine and uracil/thymine nucleotides, preferably the hexamer sequence AAUAAA. Other sequences, preferably hexamer sequences, are also conceivable. Polyadenylation typically occurs during processing of a pre-mRNA (also called premature-mRNA). Typically, RNA maturation (from pre-mRNA to mature mRNA) comprises the step of polyadenylation.

5′-cap structure: A 5′-cap is typically a modified nucleotide (cap analogue), particularly a guanine nucleotide, added to the 5′-end of an mRNA molecule. Preferably, the 5′-cap is added using a 5′-5′-triphosphate linkage (also named m7GpppN). Further examples of 5′-cap structures include glyceryl, inverted deoxy abasic residue (moiety), ′,5 methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3-phosphate, 3 phosphorothioate, phosphorodithioate, or bridging or non-bridging methylphosphonate moiety. These modified 5′-cap structures may be used in the context of the present invention to modify the mRNA sequence of the inventive composition. Further modified 5′-cap structures which may be used in the context of the present invention are CAP1 (additional methylation of the ribose of the adjacent nucleotide of m7GpppN), CAP2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse CAP analogue), modified ARCA (e.g. phosphothioate modified ARCA), inosine, Nl-methyl-guanosine, 2′-fluoro-guanosine, 7-deaza-guanosine, 8-oxo-guanosine, 2-amino-guanosine, LNA-guanosine, and 2-azido-guanosine.

Vaccine: A vaccine is typically understood to be a prophylactic or therapeutic material providing at least one antigen or antigenic function. The antigen or antigenic function may stimulate the body's adaptive immune system to provide an adaptive immune response. An antigen-providing mRNA in the context of the invention may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g. a fusion protein that consist of two or more epitopes, peptides or proteins derived from the same or different virus-proteins, wherein the epitopes, peptides or proteins may be linked by linker sequences.

Adjuvant component: An adjuvant or an adjuvant component in the broadest sense is typically a (e.g. pharmacological or immunological) agent or composition that may modify, e.g. enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of antigens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immunopotentiators, antigenic delivery systems or even combinations thereof. In the context of the present invention, an adjuvant and an immunostimulatory RNA (isRNA), such as a mRNA vaccine as generally described herein, may be a pharmaceutical composition.

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), tRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

In certain embodiment, the invention may encompass the in vitro production of artificial mRNA as well as wild-type mRNA: An artificial mRNA (sequence) may typically be understood to be an mRNA molecule, that does not occur naturally. In other words, an artificial mRNA molecule may be understood as a non-natural mRNA molecule. Such mRNA molecule may be non-natural due to its individual sequence (which does not occur naturally) and/or due to other modifications, e.g. structural modifications of nucleotides which do not occur naturally. Typically, artificial mRNA molecules may be designed and/or generated by genetic engineering methods to correspond to a desired artificial sequence of nucleotides (heterologous sequence). In this context an artificial sequence is usually a sequence that may not occur naturally, i.e. it differs from the wild type sequence by at least one nucleotide. The term “wild type” may be understood as a sequence occurring in nature. Further, the term “artificial nucleic acid molecule” is not restricted to mean “one single molecule” but is, typically, understood to comprise an ensemble of identical molecules. Accordingly, it may relate to a plurality of identical molecules contained in an aliquot.

In certain embodiment, the invention may encompass the in vitro production of bi-/multicistronic mRNA: mRNA, that typically may have two (bicistronic) or more (multicistronic) open reading frames (ORF) (coding regions or coding sequences). An open reading frame in this context is a sequence of several nucleotide triplets (codons) that can be translated into a peptide or protein. Translation of such an mRNA yields two (bicistronic) or more (multicistronic) distinct translation products (provided the ORFs are not identical). For expression in eukaryotes such mRNAs may for example comprise an internal ribosomal entry site (IRES) sequence.

In one embodiment, the in vitro produce mRNA configured to be translated to form a peptide, and preferably in a host organism, such as a mammal or human subject in need thereof. A peptide is a polymer of amino acid monomers. Usually the monomers are linked by peptide bonds. The term “peptide” does not limit the length of the polymer chain of amino acids. In some embodiments of the present invention a peptide may for example contain less than 50 monomer units. Longer peptides are also called polypeptides, typically having 50 to 600 monomeric units, more specifically 50 to 300 monomeric units.

In one embodiment, the in vitro methods described herein may produce a stabilized polynucleotide, preferably a stabilized mRNA: A stabilized nucleic acid, preferably mRNA typically, exhibits a modification increasing resistance to in vivo degradation (e.g. degradation by an exo- or endo-nuclease) and/or ex vivo degradation (e.g. by the manufacturing process prior to vaccine administration, e.g. in the course of the preparation of the vaccine solution to be administered). Stabilization of RNA can, e.g., be achieved by providing a 5′-CAP-Structure, a Poly(A)-Tail, or any other UTR-modification. It can also be achieved by chemical modification or modification of the G/C-content of the nucleic acid. Various other methods are known in the art and conceivable in the context of the invention.

A “pharmaceutical composition” may include a vaccine of the invention and an agent, e.g. a carrier, that may typically be used within a pharmaceutical composition or vaccine for facilitating administering of the components of the pharmaceutical composition or vaccine to an individual.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “about” or “approximately” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” or “approximately” will depend upon the particular system under study. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

SEQUENCE LISTING

SEQ ID NO. 1
AA/DNA
RNA Polymerase
T7 Bacteriophage
MNTINIAKNDFSDIELAAIPFNTLADHYGERLAREQLALEHESYEMGEARFRKMFERQLKAGEVADNAAAKPLITTL
LPKMIARINDWFEEVKAKRGKRPTAFQFLQEIKPEAVAYITIKTTLACLTSADNTTVQAVASAIGRAIEDEARFGRI
RDLEAKHFKKNVEEQLNKRVGHVYKKAFMQVVEADMLSKGLLGGEAWSSWHKEDSIHVGVRCIEMLIESTGMVSLHR
QNAGVVGQDSETIELAPEYAEAIATRAGALAGISPMFQPCVVPPKPWTGITGGGYWANGRRPLALVRTHSKKALMRY
EDVYMPEVYKAINIAQNTAWKINKKVLAVANVITKWKHCPVEDIPAIEREELPMKPEDIDMNPEALTAWKRAAAAVY
RKDKARKSRRISLEFMLEQANKFANHKAIWFPYNMDWRGRVYAVSMFNPQGNDMTKGLLTLAKGKPIGKEGYYWLKI
HGANCAGVDKVPFPERIKFIEENHENIMACAKSPLENTWWAEQDSPFCFLAFCFEYAGVQHHGLSYNCSLPLAFDGS
CSGIQHFSAMLRDEVGGRAVNLLPSETVQDIYGIVAKKVNEILQADAINGTDNEVVTVTDENTGEISEKVKLGTKAL
AGQWLAYGVTRSVTKRSVMTLAYGSKEFGFRQQVLEDTIQPAIDSGKGLMFTQPNQAAGYMAKLIWESVSVTVVAAV
EAMNWLKSAAKLLAAEVKDKKTGEILRKRCAVHWVTPDGFPVWQEYKKPIQTRLNLMFLGQFRLQPTINTNKDSEID
AHKQESGIAPNFVHSQDGSHLRKTVVWAHEKYGIESFALIHDSFGTIPADAANLFKAVRETMVDTYESCDVLADFYD
QFADQLHESQLDKMPALPAKGNLNLRDILESDFAFA
SEQ ID NO. 2
Amino Acid
mRNA_triPase
[INSERT]¹
YRNVPIWAQKWKPTIKALQSINVKDLKIDPSFLNIIPDDDLTKSVQDWVYATIYSIAPELRSFI
ELEMKFGVIIDAKGPDRVNPPVSSQCVFTELDAHLTPNIDASLFKELSKYIRGISEVTENTGKF
SIIESQTRDSVYRVGLSTQRPRFLRMSTDIKTGRVGQFIEKRHVAQLLLYSPKDSYDVKISLNL
ELPVPDNDPPEKYKSQSPISERTKDRVSYIHNDSCTRIDITKVENHNQNSKSRQSETTHEVELE
INTPALLNAFDNITNDSKEYASLIRTFLNNGTIIRRKLSSLSYEIFEGSKKVM
SEQ ID NO. 3
Amino Acid
mRNA guanylyltransferase
Chlorella virus
MVPPTINTGKNITTERAVLTLNGLQIKLHKVVGESRDDIVAKMKDLAMDDHKFPRLPGPNPVSI
ERKDFEKLKQNKYVVSEKTDGIRFMMFFTRVFGFKVCTIIDRAMTVYLLPFKNIPRVLFQGSIF
DGELCVDIVEKKFAFVLFDAVVVSGVTVSQMDLASRFFAMKRSLKEFKNVPEDPAILRYKEWIP
LEHPTIIKDHLKKANAIYHTDGLIIMSVDEPVIYGRNFNLFKLKPGTHHTIDFIIMSEDGTIGI
FDPNLRKNVPVGKLDGYYNKGSIVECGFADGTWKYIQGRSDKNQANDRLTYEKTLLNIEENITI
DELLDLFKWE
SEQ ID NO. 4
Amino Acid
mRNA cap guanine-N7 methyltransferase
Encephalitozoon cuniculi
MEGKKEEIREHYNSIRERGRESRQRSKTINIRNANNEIKACLIRLYTKRGDSVLDLGCGKGGDL
LKYERAGIGEYYGVDIAEVSINDARVRARNMKRRFKVFFRAQDSYGRHMDLGKEFDVISSQFSF
HYAFSTSESLDIAQRNIARHLRPGGYFIMTVPSRDVILERYKQGRMSNDFYKIELEKMEDVPME
SVREYRFTLLDSVNNCIEYFVDFTRMVDGFKRLGLSLVERKGFIDFYEDEGRRNPELSKKMGLG
CLTREESEWGIYEVVVFRKLVPESDA
SEQ ID NO. 5
Amino Acid
Poly(A) Polymerase
Saccharomyces cerevisiae
MSSQKVFGITGPVSTVGATAAENKLNDSLIQELKKEGSFETEQETANRVQVLKILQELAQRFVY
EVSKKKNMSDGMARDAGGKIFTYGSYRLGVHGPGSDIDTLVVVPKHVTREDFFTVFDSLLRERK
ELDEIAPVPDAFVPIIKIKFSGISIDLICARLDQPQVPLSLTLSDKNLLRNLDEKDLRALNGTR
VTDEILELVPKPNVFRIALRAIKLWAQRRAVYANIFGFPGGVAWAMLVARICQLYPNACSAVIL
NRFFIILSEWNWPQPVILKPIEDGPLQVRVWNPKIYAQDRSHRMPVITPAYPSMCATHNITEST
KKVILQEFVRGVQITNDIFSNKKSWANLFEKNDFFFRYKFYLEITAYTRGSDEQHLKWSGLVES
KVRLLVMKLEVLAGIKIAHPFTKPFESSYCCPTEDDYEMIQDKYGSHKTETALNALKLVTDENK
EEESIKDAPKAYLSTMYIGLDFNIENKKEKVDIHIPCTEFVNLCRSFNEDYGDHKVFNLALRFV
KGYDLPDEVFDENEKRPSKKSKRKNLE
SEQ ID NO. 6
Amino Acid
VP39
Vaccinia Virus
MDVVSLDKPFMYFEEIDNELDYEPESANEVAKKLPYQGQLKLLLGELFFLSKLQRHGILDGATV
VYIGSAPGTHIRYLRDHFYNLGVIIKWMLIDGRHHDPILNGLRDVTLVTRFVDEEYLRSIKKQL
HPSKIILISDVRSKRGGNEPSTADLLSNYALQNVMISILNPVASSLKWRCPFPDQWIKDFYIPH
GNKMLQPFAPSYSAEMRLLSIYTGENMRLTRVTKSDAVNYEKKMYYLNKIVRNKVVVNFDYPNQ
EYDYFHMYFMLRTVYCNKTFPTTKAKVLFLQQSIFRFLNIPTTSTEKVSHEPIQRKISSKNSMS
KNRNSKRSVRSNK
SEQ ID NO. 7
Amino Acid
VP55
Vaccinia Virus
MNRNPDQNTLPNITLKIIETYLGRVPSVNEYHMLKLQARNIQKITVFNKDIFVSLVKKNKKRFF
SDVNTSASEIKDRILSYFSKQTQTYNIGKLFTIIELQSVLVTTYTDILGVLTIKAPNVISSKIS
YNVTSMEELARDMLNSMNVAVIDKAKVMGRHNVSSLVKNVNKLMEEYLRRHNKSCICYGSYSLY
LINPNIRYGDIDILQTNSRTFLIDLAFLIKFITGNNIILSKIPYLRNYMVIKDENDNHIIDSFN
IRQDTMNVVPKIFIDNIYIVDPTFQLLNMIKMFSQIDRLEDLSKDPEKFNARMATMLEYVRYTH
GIVFDGKRNNMPMKCIIDENNRIVTVTTKDYFSFKKCLVYLDENVLSSDILDLNADTSCDFESV
TNSVYLIHDNIMYTYFSNTILLSDKGKVHEISARGLCAHILLYQMLTSGEYKQCLSDLLNSMMN
RDKIPIYSHTERDKKPGRHGFINIEKDIIVF
¹Note: Add organism that mRNA-tripase is derived from

1. A continuous-flow recombinant system for producing messenger RNA (mRNA) polynucleotides in vitro comprising:
a continuous-flow bioreactor having:
at least one continuous-flow reaction chamber configured to hold an input reaction mixture having a DNA template; and
at least one continuous-flow conduit configured hold and circulate a feed solution and to further configured to be in fluid communication with said continuous-flow reaction chamber through a series of conduit apertures forming a gradient between said input reaction mixture and said feed solution;
wherein said input reaction mixture and said feed solution contain all necessary components for the in vitro generation of a target mRNA transcribed from said DNA template;
a protein removal component configured to remove the protein fraction of said mRNA output;
a DNA removal component configured to remove the DNA fraction of said mRNA output; and
a nucleotide precipitation component configured to remove the nucleotide triphosphates (NTP) fraction of said mRNA output.

2. The system of claim 1, wherein said DNA template comprises a linear DNA template, or a circular DNA template.

3. The system of claim 1, wherein said DNA template encodes an antigenic polypeptide.

4. (canceled)

5. The system of claim 1, wherein said input reaction mixture comprises one or more components selected from the group consisting of:
a first quantity of isolated RNA polymerase (RNAP) enzyme;
a quantity of a reaction buffer; and
optionally an initial quantity of isolated nucleotide triphosphates (NTPs).

6. The system of claim 1, wherein said feed solution comprises one or more components selected from the group consisting of:
a first quantity of isolated NTPs;
a quantity of a reaction buffer;
optionally the components of an inorganic polyphosphate energy-regeneration system; and
optionally one or more co-factors for the production of mRNA polynucleotides.

7. The system of claim 1, wherein said protein removal component comprises a protein affinity column configured to remove the protein fraction from said mRNA output.

8. The system of claim 1, wherein said DNA removal component comprises a DNA affinity column configured to remove the protein fraction from said mRNA output.

9. The system of claim 1, wherein said nucleotide precipitation component comprises an alcohol precipitation system configured to isolate the target mRNA from said mRNA output.

10-13. (canceled)

14. The system of claim 1, and further comprising an input reservoir coupled with an input valve configured to allow real-time injection of feed solution into the continuous-flow conduit.

15. The system of claim 1, and further comprising an output reservoir coupled with an output valve configured to allow extraction of the mRNA output from the continuous-flow reaction chamber.

16. A batch-fed recombinant system for producing messenger RNA (mRNA) polynucleotides in vitro comprising:
batch-fed reaction chamber configured to hold an input reaction mixture having a DNA template and a feed solution wherein said input reaction mixture and said feed solution contain all necessary components for the in vitro generation of a target mRNA transcribed from said DNA template;
a protein removal component configured to remove the protein fraction of said mRNA output;
a DNA removal component configured to remove the DNA fraction of said mRNA output; and
a nucleotide precipitation component configured to remove the nucleotide triphosphates (NTP) fraction of said mRNA output.

17. The system of claim 16, wherein said DNA template comprises a linear DNA template, or a circular DNA template.

18. The system of claim 16, wherein said DNA template encodes an antigenic polypeptide.

19. (canceled)

20. The system of claim 16, wherein said input reaction mixture comprises one or more components selected from the group consisting of:
a first quantity of isolated RNA polymerase (RNAP) enzyme;
a quantity of a reaction buffer; and
optionally an initial quantity of isolated nucleotide triphosphates (NTPs).

21. The system of claim 16, wherein said feed solution comprises one or more components selected from the group consisting of:
a first quantity of isolated NTPs;
a quantity of a reaction buffer;
optionally the components of an inorganic polyphosphate energy-regeneration system; and
optionally one or more co-factors for the production of mRNA polynucleotides.

22. The system of claim 16, wherein said protein removal component comprises a protein affinity column configured to remove the protein fraction from said mRNA output.

23. The system of claim 16, wherein said DNA removal component comprises a DNA affinity column configured to remove the protein fraction from said mRNA output.

24. The system of claim 16, wherein said nucleotide precipitation component comprises an alcohol precipitation system configured to isolate the target mRNA from said mRNA output.

25-27. (canceled)

28. The system of claim 16, and further comprising an input reservoir coupled with said batch-fed reaction chamber.

29. The system of claim 16, and further comprising an output reservoir coupled with said batch-fed reaction chamber.

30-45. (canceled)


SEQUENCE LISTING (7 sequences)

SEQ ID NO: 1 - protein, 883 aa, T7 Bacteriophage
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 309 aa, Saccharomyces cerevisiae
YRNVPIWAQKWKPTIKALQSINVKDLKIDPSFLNIIPDDDLTKSVQDWVYATIYSIAPELRSFIELEMKFGVIIDAKGPDRVNPPVSSQCVFTELDAHLTPNIDASLFKELSKYIRGISEVTENTGKFSIIESQTRDSVYRVGLSTQRPRFLRMSTDIKTGRVGQFIEKRHVAQLLLYSPKDSYDVKISLNLELPVPDNDPPEKYKSQSPISERTKDRVSYIHNDSCTRIDITKVENHNQNSKSRQSETTHEVELEINTPALLNAFDNITNDSKEYASLIRTFLNNGTIIRRKLSSLSYEIFEGSKKVM

SEQ ID NO: 3 - protein, 330 aa, Chlorella virus
MVPPTINTGKNITTERAVLTLNGLQIKLHKVVGESRDDIVAKMKDLAMDDHKFPRLPGPNPVSIERKDFEKLKQNKYVVSEKTDGIRFMMFFTRVFGFKVCTIIDRAMTVYLLPFKNIPRVLFQGSIFDGELCVDIVEKKFAFVLFDAVVVSGVTVSQMDLASRFFAMKRSLKEFKNVPEDPAILRYKEWIPLEHPTIIKDHLKKANAIYHTDGLIIMSVDEPVIYGRNFNLFKLKPGTHHTIDFIIMSEDGTIGIFDPNLRKNVPVGKLDGYYNKGSIVECGFADGTWKYIQGRSDKNQANDRLTYEKTLLNIEENITIDELLDLFKWE

SEQ ID NO: 4 - protein, 283 aa, Encephalitozoon cuniculi
MEGKKEEIREHYNSIRERGRESRQRSKTINIRNANNFIKACLIRLYTKRGDSVLDLGCGKGGDLLKYERAGIGEYYGVDIAEVSINDARVRARNMKRRFKVFFRAQDSYGRHMDLGKEFDVISSQFSFHYAFSTSESLDIAQRNIARHLRPGGYFIMTVPSRDVILERYKQGRMSNDFYKIELEKMEDVPMESVREYRFTLLDSVNNCIEYFVDFTRMVDGFKRLGLSLVERKGFIDFYEDEGRRNPELSKKMGLGCLTREESEVVGIYEVVVFRKLVPESDA

SEQ ID NO: 5 - protein, 539 aa, Saccharomyces cerevisiae
MSSQKVFGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEKNDFFFRYKFYLEITAYTRGSDEQHLKWSGLVESKVRLLVMKLEVLAGIKIAHPFTKPFESSYCCPTEDDYEMIQDKYGSHKTETALNALKLVTDENKEEESIKDAPKAYLSTMYIGLDFNIENKKEKVDIHIPCTEFVNLCRSFNEDYGDHKVFNLALRFVKGYDLPDEVFDENEKRPSKKSKRKNLE

SEQ ID NO: 6 - protein, 333 aa, Vaccinia Virus
MDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTEKVSHEPIQRKISSKNSMSKNRNSKRSVRSNK

SEQ ID NO: 7 - protein, 479 aa, Vaccinia Virus
MNRNPDQNTLPNITLKIIETYLGRVPSVNEYHMLKLQARNIQKITVFNKDIFVSLVKKNKKRFFSDVNTSASEIKDRILSYFSKQTQTYNIGKLFTIIELQSVLVTTYTDILGVLTIKAPNVISSKISYNVTSMEELARDMLNSMNVAVIDKAKVMGRHNVSSLVKNVNKLMEEYLRRHNKSCICYGSYSLYLINPNIRYGDIDILQTNSRTFLIDLAFLIKFITGNNIILSKIPYLRNYMVIKDENDNHIIDSFNIRQDTMNVVPKIFIDNIYIVDPTFQLLNMIKMFSQIDRLEDLSKDPEKFNARMATMLEYVRYTHGIVFDGKRNNMPMKCIIDENNRIVTVTTKDYFSFKKCLVYLDENVLSSDILDLNADTSCDFESVTNSVYLIHDNIMYTYFSNTILLSDKGKVHEISARGLCAHILLYQMLTSGEYKQCLSDLLNSMMNRDKIPIYSHTERDKKPGRHGFINIEKDIIVF